(19) United States
(12) Plant Patent Application Publication
(10) Pub. No.: US 2003/0051277 P1
(43) Pub. Date: Mar. 13, 2003

(54) Floribunda rose plant named 'Meizebul'

(76) Inventor: Alain A. Meilland, Antibes (FR)

Correspondence Address: BURNS DOANE SWECKER & MATHIS L L P, POST OFFICE BOX 1404, ALEXANDRIA, VA, 22313-1404, US

(21) Appl. No.: 09/947,568

(22) Filed: Sep. 7, 2001

(51) Int. Cl. A01H 5/00

(52) U.S. Cl. PLT/148

(57) ABSTRACT

A new and distinct variety of Floribunda rose plant is provided which abundantly forms clusters of attractive double lightly fragrant blossoms that possess a very stable pink coloration. Such blossoms display a light fragrance. The plant exhibits a regular, compact and bushy growth habit, forms very dense dark green semi-glossy foliage, and possesses very good disease resistance. The new variety is particularly well suited for providing colorful ornamentation in the landscape.

### SUMMARY OF THE INVENTION

The new Rosa hybrida variety of Floribunda rose plant was created by artificial pollination wherein two parents were crossed which previously had been studied in the hope that they would contribute the desired characteristics. The female parent (i.e., the seed parent) of the new variety was the product of the ‘Delceli’ variety (non-patented in the United States). The male parent (ie., the pollen parent) was the ‘Meidragelac’ variety (non-patented in the United States). The parentage of the new variety can be summarized as follows:

‘Delceli’×‘Meidragelac’.

The seeds resulting from the above pollination were sown and small plants were obtained which were physically and biologically different from each other. Selective study resulted in the identification of a single plant of the new variety.

It was found that the new variety of Floribunda rose plant of the present invention possesses the following combination of characteristics:

(a) forms on an abundant basis clusters of attractive double lightly fragrant blossoms that are of a very stable pink coloration,

(b) exhibits a regular, compact and bushy growth habit,

(c) forms attractive very dense dark green semi-glossy foliage, and

(d) is particularly well suited for growing as ornamentation in parks and gardens.

The new variety well meets the needs of the horticultural industry and is particularly well suited for growing in the landscape.

The new variety of the present invention can be readily distinguished from its parental varieties. For instance, the ‘Delceli’ variety is lesser floriferous and forms salmon flowers with a silver reverse having no fragrance and approximately 23 petals. The ‘Meidragelac’ variety forms smaller flowers having approximately 30 petals that are orange-red with a lighter reverse.

The new variety has been found to undergo asexual propagation in France by a number of routes, including budding, grafting, and cuttage. Asexual propagation by the above-mentioned techniques in France has shown that the characteristics of the new variety are stable and are strictly transmissible by such asexual propagation from one generation to another.

The new variety has been named the ‘Meizebul’ variety.

### BRIEF DESCRIPTION OF THE PHOTOGRAPH

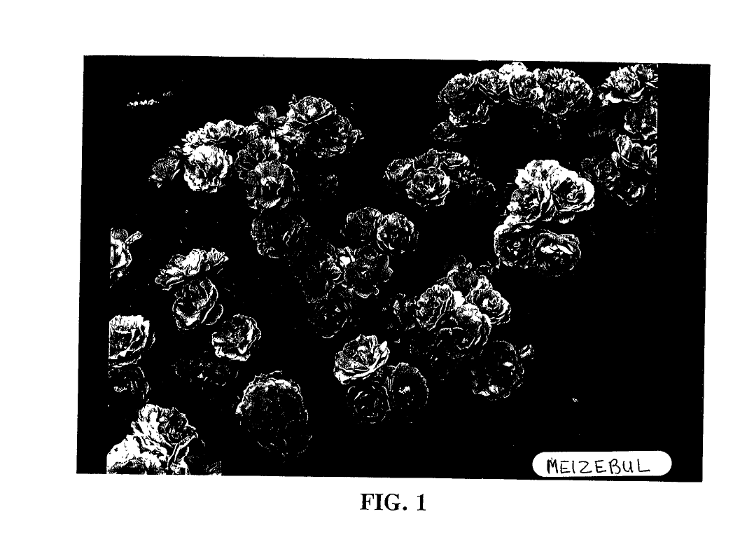

The accompanying FIG. 1 shows as nearly true as it is reasonably possible to make the same, in a color illustration of this character, typical plants of the new variety with foliage and blossoms in various stages of opening. The rose plants of the new variety were two years of age and were observed during June while budded on Rosa froebelli understock and growing outdoors at Le Cannet des Maures, Var, France.

### DETAILED DESCRIPTION

The chart used in the identification of the colors is that of The Royal Horticultural Society (R.H.S. Colour Chart). The description is based on the observation of two year-old plants during September while budded on Rosa froebelli understock and growing outdoors at Le Cannet des Maures, Var, France. The coloration in common terms precedes reference to the chart in some instances.

Class: Floribunda.

Plant:

height.—approximately 90 to 100 cm on average at the end of the growing season.

habit.—regular, compact and bushy.

Branches:

color.—young stems: medium green.

adult wood.—medium green.

thorns.—size: medium. quantity: moderately sparse. shape: slightly concave.

Leaves:

stipules.—adnate, pectinate, and narrow.

leaflets.—number: 3, 5, and 7. shape: ovate with a narrow tip and an obtuse base. serration: regular. texture: frmn. general appearance: very dense, dark green, and semi-glossy foliage.

Inflorescence:

number of flowers.—usually 6 to 15 flowers per stem.

peduncle.—medium green with anthocyanin coloration, medium in size, and approximately 5 cm in length on average.

sepals.—upper surface: tomentose and light green with reddish anthocyanin coloration. under surface: medium green. configuration: elongated.

buds.—shape: conical. size: medium. length: approximately 2 cm on average. color upon opening: upper surface: Scarlet, near Red Group 43B and 43C. under surface: Neyron Rose, near Red Group 55B and 55C.

flower.—shape: double and flattened when fully open. diameter: commonly approximately 9 cm on average. color (when opening begins): upper surface: Scarlet, near Red Group 43B and 43C. under surface: Neyron Rose, near Red Group 55B and 55C. color (when blooming): upper surface: Carmine Rose, near Red Group 52C. under surface: Neyron Rose, near Red Group 55B and 55C. color (at end of opening): upper surface: Carmine Rose, near Red Group 52C. under surface: Neyron Rose, near Red Group 55B and 55C. fragrance: light. lasting quality: very long on the plant. petal shape: cordate. petal number: approximately 24 to 28 on average. receptacle: medium green.

Development

vegetation.—very strong and vigorous.

blooming.—abundant.

resistance to diseases.—very good.

aptitude to bear fruit.—very weak.

I claim:

1. A new and distinct variety of Floribunda rose plant characterized by the following combination of characteristics:
(a) forms on an abundant basis clusters of attractive double lightly fragrant blossoms that are of a very stable pink coloration,
(b) exhibits a regular, compact and bushy growth habit,
(c) forms attractive very dense dark green semi-glossy foliage, and
(d) is particularly well suited for growing as ornamentation in parks and gardens; substantially as herein shown and described.
